(19) United States
(12) Patent Application Publication
(10) Pub. No.: US 2002/0034526 A1
(43) Pub. Date: Mar. 21, 2002

(54) Composition comprising a hydrophilic component and a combination of a volatile compound and a phenylated silicone oil, and use thereof

(75) Inventors: Isabelle Agostini, Chatenay Malabry (FR); Dolores Miguel-Colombel, L'Hay-Les-Roses (FR)

Correspondence Address: FINNEGAN, HENDERSON, FARABOW, GARRETT &, DUNNER LLP, 1300 I STREET, NW, WASHINGTON, DC, 20005, US

(73) Assignee: L'Oreal, Paris (FR)

(21) Appl. No.: 09/951,735

(22) Filed: Sep. 14, 2001

Related U.S. Application Data
(63) Continuation of application No. 09/413,825, filed on Oct. 7, 1999, now abandoned.
Application No. 09/413,825 is a division of application No. 08/896,220, filed on Jul. 17, 1997.

(30) Foreign Application Priority Data
Jul. 17, 1996 (FR) 96-08956

(51) Int. Cl. A61K 7/021; A61K 7/025; A61K 7/035; A61K 7/06; A61K 9/00; A61K 6/00; A61K 7/00

(52) U.S. Cl. 424/401; 424/63; 424/64; 424/69; 424/70.1; 424/70.7

(57) ABSTRACT

A cosmetic, dermatological, hygiene and/or pharmaceutical composition comprising at least one hydrophilic component and a fatty phase which comprises at least one volatile compound and at least one phenylated silicone oil. The composition is, in particular, a composition having improved staying power. The invention also relates to the use of the combination of a volatile compound and a phenylated silicone oil to limit the transfer and/or migration of such a cosmetic, dermatological, hygiene and/or pharmaceutical composition comprising a hydrophilic compound.

The present invention relates to a composition, in particular a cosmetic composition, which may be in the form of a stick or a soft paste and which may be used to care for and/or make up the skin, semi-mucous membranes and/or mucous membranes, and in particular the lips of the face.

The solid, fatty cosmetic or pharmaceutical compositions usually used especially in the form of pencils, for example lipstick pencils, have the drawback of being relatively non-moisturizing. To overcome this drawback, it has been attempted to introduce water therein by preparing emulsified products. Mention may be made in this respect of French patent application FR 2,237,615, which describes a water-in-oil emulsion containing from 1 to 50% by weight of water. However, this type of composition poses problems of evaporation of the water. The composition described in that patent application also contains from 1 to 10% of a polyhydroxylated compound in order to obtain uniform distribution of the dye substances in the water-in-oil emulsion.

The problem of evaporation of the water from solid dispersions has been solved according to European patent application EP 374,332 by the incorporation of a silicone oil and a polyorganosiloxane modified with a polyoxyalkylene. The compositions thus modified may also contain polyols in a low amount as additional wetting agents.

Japanese patent application JP 01-143,812 describes the incorporation of a large amount of polyvalent alcohols into a solid emulsion for cosmetic use by means of a silicone oil and a denatured polyoxyalkylene polyorganosiloxane. However, it is difficult to keep the formulation homogeneous with that system.

Lastly, application EP 524,892 is known, the aim of which is to overcome the drawbacks described above by proposing a specific process which makes it possible to obtain a solid, anhydrous dispersion which comprises fatty substances and a polyhydric alcohol in a large amount.

However, it was observed that when all of these compositions are applied to the skin, mucous membranes or semi-mucous membranes, they have the drawback of transferring. This is understood to mean that the composition is liable to be deposited, at least partly, on certain supports with which it is placed in contact, such as, for example, a glass, clothing or the skin. On being deposited, the composition leaves a trace on the support. This results in mediocre persistence of the composition on the skin or the mucous membranes, making it necessary to repeat its application regularly.

Another drawback of these compositions lies in the problem of migration. This is because it has been seen that certain compositions have a tendency to spread inside the wrinkles and/or fine lines of the skin, in the case of foundations; in the fine lines around the lips, in the case of lipsticks; in the folds of the eyelid, in the case of eyeshadows.

Especially in the case of eyeshadows, the appearance of lines in the make-up which are generated by the movements of the eyelids has also been observed. All of these phenomena produce an unpleasant-looking make-up effect which it would obviously be desirable to avoid.

These drawbacks, which are generally present for common lipstick compositions, are even more pronounced when the composition comprises polyols. This is due in particular to the fact that polyols are sticky and viscous compounds which tend to adhere to and be deposited on the support with which they are placed in contact, leaving a trace thereon.

There is thus a need for a composition, in particular a cosmetic composition, which transfers little or not at all, that is to say a “non-transferring” composition, while at the same time possessing good moisturizing and/or emollient properties.

In particular, there is a need for a composition which is capable of comprising hydrophilic compounds such as polyols in a large amount, while at the same time retaining very good staying power, that is to say not transferring and not marking, or marking only very little, a support with which it might come into contact, and not migrating over time.

The aim of the present invention is thus to propose such a composition, which transfers little or not at all, although it comprises hydrophilic compounds.

The subject of the invention is thus a cosmetic, dermatological, hygiene and/or pharmaceutical composition comprising a hydrophilic component and a fatty phase which comprises at least one volatile compound and at least one phenylated silicone oil.

The subject of the invention is also the use of the combination of a volatile compound and a phenylated silicone oil in order to limit, decrease and/or eliminate transfer and/or migration, and/or in order to improve the staying power, of a cosmetic, dermatological, hygiene and/or pharmaceutical composition comprising a hydrophilic component.

The subject of the invention is also a process for limiting, decreasing and/or eliminating the transfer of a cosmetic, dermatological, hygiene and/or pharmaceutical composition comprising a hydrophilic component, in particular a make-up and/or care composition for the skin, mucous membranes, semi-mucous membranes and/or the exoskeleton, this process comprising including in the composition at least one volatile compound and at least one phenylated silicone oil.

It has been observed that the composition according to the invention can be applied easily to the skin and that it affords, inter alia, a make-up of light texture which remains comfortable to wear throughout the day. Furthermore, the composition according to the invention possesses excellent emollient properties when applied to the skin.

The composition according to the invention finds a particularly advantageous application in the field of caring for and/or making up the skin, mucous membranes, semi-mucous membranes and the exoskeleton. The term mucous membrane is understood in particular to refer to the inner part of the lower eyelid; the term semi-mucous membranes is understood to refer more particularly to the lips of the face, the term exoskeleton is understood to refer to the eyelashes, the eyebrows, the hair and the nails. Thus, the invention finds an application most particularly in the field of make-up products for the lips of the face and also lipcare products, as well as in the field of make-up and care products for the skin such as foundations, self-tanning agents or antisun products.

The composition according to the invention thus comprises at least one hydrophilic component, which may preferably be a polyhydric alcohol and/or optionally a thickened or even gelled, aqueous phase.

The polyhydric alcohol may be a compound having from 2 to 8 carbon atoms and from 2 to 6 hydroxyl functions, such as ethylene glycol, glycerol, 1,2-propanediol, diglycerol, erythritol, arabitol, adonitol, sorbitol and dulcitol. The polyhydric alcohol may also be a polyether alcohol with an average molecular weight of 150 to 600, such as polyethylene glycol 300 and polyglycerol 500, wherein the molecular weight is a weight-average molecular weight. A mixture of polyhdyric alcohols may also be used.

The thickened or gelled aqueous phase may comprise water and/or a flower water such as cornflower water, and/or a mineral water such as Eau de Vittel, Eau de Lucas or Eau de La Roche Posay.

It may be thickened by the presence of any thickener known to those skilled in the art. Mention may be made in particular of:

extracts of algae such as agar-agar, carrageenans and alginates,

seed extracts such as extracts of carob or of guar,

fruit extracts, in particular pectin,

plant exudates such as gum arabic, gum tragacanth, karaya gum and ghatti gum,

cellulose derivatives such as carboxy-methylcellulose,

gelling agents of animal origin such as gelatin or caseinates,

micro-organism exudates such as xanthan gum,

synthetic gelling agents such as acrylic polymer derivatives (CARBOMER,

CARBOPOL, PEMULEN) or silicon derivatives (LAPONITE, LAPOMER or VEEGUM).

The thickener may be present in the aqueous phase in a proportion of 0.2 to 10% by weight relative to the aqueous phase.

In addition, the hydrophilic component may comprise water-soluble additives, in particular vitamins, cosmetic and/or pharmaceutical active agents, dyes, amino acids and UV screening agents.

The hydrophilic component may represent 4 to 40% by weight of the composition, preferably 8 to 25% by weight.

Preferably, the hydrophilic component is dispersed in at least a part of the fatty phase, before being mixed with the rest of the constituents of the composition. When the hydrophilic component comprises at least one polyhydric alcohol, the particle size of the alcohol is preferably less than or equal to one micron.

In order to obtain a finer dispersion of the hydrophilic component in the composition, at least one surfactant may be added to the composition. This surfactant may be anionic or nonionic, preferably with an HLB of less than 10, more preferably less than 5. Mention may be made in particular of silicone derivatives such as silicone surfactants, or any other common surfactant such as lecithins, succinylglycerides, alkyl phosphates, magnesium lanolate and mixtures thereof.

The composition according to the invention thus also comprises a fatty phase, which comprises in particular volatile compounds. These volatile compounds may be selected in particular from hydrocarbon oils and/or silicone oils, which are cyclic or linear, alone or as a mixture.

In the present description, the term volatile compound is understood to refer to any compound which can evaporate through contact with the skin. Preferably, volatile oils whose flash point is sufficiently high to allow them to be used in formulation, and sufficiently low to obtain the desired evanescent effect are used. Volatile oils whose flash point is about 40 to 100° C. are preferably used.

Among the volatile silicone oils, mention may be made, alone or as a mixture, of

volatile cyclic silicones having from 3 to 8 and preferably from 4 to 6 silicon atoms. These are, for example, cyclotetradimethylsiloxane, cyclopenta-dimethylsiloxane or cyclohexadimethylsiloxane;

cyclocopolymers of the dimethylsiloxane/methylalkylsiloxane type, such as Silicone FZ 3109 sold by the company Union Carbide, which is a dimethylsiloxane/methyloctylsiloxane cyclocopolymer;

volatile linear silicones having from 2 to 9 silicon atoms. These are, for example, hexamethyl-disiloxane or a PDMS of low viscosity (1 cSt). Mention may also be made of alkyltrisiloxanes such as hexylheptamethyltrisiloxane or octylheptamethyl-trisiloxane.

Among the volatile hydrocarbon oils, mention may be made of isoparaffins and in particular isododecane.

The composition according to the invention may comprise from 8 to 70% by weight, preferably from 15 to 55%, of volatile compounds relative to the total weight of the composition.

The composition according to the invention also comprises at least one phenylated silicone oil, in particular a polyphenylmethylsiloxane or a phenyltrimethicone, or a mixture of different phenylated silicone oils.

In particular, these oils may correspond to the following formula:

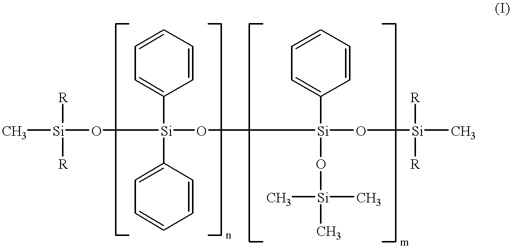

in which

R is a C₁-C₃₀ alkyl radical, an aryl radical or an aralkyl radical,

n is an integer from 0 to 100,

m is an integer from 0 to 100, with the proviso that the sum m+n is from 1 to 100.

Preferably, R is a methyl, ethyl, propyl, isopropyl, decyl, dodecyl or octadecyl radical or alternatively a phenyl, tolyl, benzyl or phenethyl radical.

Among these phenylated oils, mention may be made of the oil BELSIL PDM1000 from Wacker, the oils DC556 or SF558 from Dow Corning, the oil ABIL AV8853 from Goldschmidt or the oil SILBIONE 70633V30 from Rhone Poulenc.

The composition according to the invention may comprise from 1 to 35% by weight, preferably from 20 to 30%, of phenylated silicone oils.

The composition according to the invention may also comprise at least one wax, which can in particular provide the composition with mechanical strength, when it is in the form of a stick. When it is in the form of a soft paste or a cast product, the composition according to the invention may comprise a lesser amount of wax.

Any wax known in the prior art may be used, among which, alone or as a mixture, mention may be made of animal, plant, mineral and synthetic waxes such as microcrystalline waxes, paraffin, petrolatum, petroleum jelly, ozokerite, montan wax, beeswax, lanolin and derivatives thereof, candelilla wax, ouricurry wax, carnauba wax, Japan wax, cocoa butter, cork fibre wax or sugar cane wax; hydrogenated oils which are solid at 25° C., fatty esters and glycerides which are solid at 25° C.; polyethylene waxes and the waxes obtained by Fischer-Tropsch synthesis; silicone waxes; fluoro waxes.

Preferably, the waxes forming part of the composition may have a melting point above about 45° C. and in particular above 55° C., and/or a needle penetration index at 25° C. preferably of from 3 to 40. The composition preferably comprises 0.5 to 30% by weight of wax, more preferably 10 to 20% by weight.

Moreover, the composition may comprise, in addition to the compounds mentioned above, the fatty substances usually used in the field of application envisaged, such as oils, gums and/or pasty fatty substances of plant, mineral, animal, synthetic or silicone origin. These fatty substances may be selected in particular in a varied manner by those skilled in the art in order to prepare a composition having the desired properties, for example in terms of consistency or texture.

In particular, mention may be made of polydimethylsiloxanes (PDMS) and alkyldimethicones, as well as silicones modified with aliphatic and/or aromatic groups, which may optionally contain fluorine, or with functional groups such as hydroxyl, thiol and/or amine groups.

Mention may also be made of liquid paraffin, liquid petroleum jelly, perhydrosqualene, groundnut oil, sweet almond oil, beauty-leaf oil, palm oil, castor oil, avocado oil, jojoba oil, olive oil or cereal germ oil; fatty acid esters; alcohols; acetylglycerides; octanoates, decanoates or ricinoleates of alcohols or of polyalcohols; fatty acid triglycerides; glycerides.

However, it has been observed that the improvement in the staying power of the composition according to the invention, as well as its absence of migration and/or transfer, may be particularly advantageous when the composition comprises less than 20% by weight of non-volatile hydrocarbon oil, preferably less than 5% by weight, or even no non-volatile hydrocarbon oils at all.

It is also possible to add to the composition according to the invention an amount of a polymer which is sufficient to stabilize it further in a humid atmosphere. Such polymers should be liposoluble and possess a very low content of hydrophilic units. Among these polymers, mention may be made of polyalkylenes (in particular polyethylenes and polybutenes), polyacrylates and silicone polymers which are compatible with fatty substances. Among the polyalkylenes, mention may be made of polybutene, in particular that sold by the company Amoco under the name INDOPOL.

The weight ratio of the polymer to the polyhydric alcohol is preferably less than 4:1, more particularly from 0.25:1 to 2:1.

The composition may also comprise a particulate phase, which may comprise pigments and/or pearlescent agents and/or fillers usually used in cosmetic compositions. The term pigments should be understood to mean white or colored, inorganic or organic particles intended to color and/or opacify the composition. The term fillers should be understood to mean colorless or white, inorganic or synthetic, lamellar or non-lamellar particles intended to impart body or rigidity to the composition. The term pearlescent agents should be understood to mean iridescent particles which reflect light, of natural or synthetic origin.

The pigments may be present in the composition in a proportion of from 0 to 15% by weight of the final composition, and preferably in a proportion of from 8 to 10%. They may be white or colored, inorganic and/or organic.

Mention may be made of titanium dioxide, zirconium dioxide or, cerium dioxide as well as zinc oxide, iron oxide or chromium oxide, ferric blue, carbon black, ultramarines, manganese pyrophosphate and certain metal powders such as those of silver or of aluminum. Mention may also be made of the lakes commonly employed to impart a make-up effect to the lips and the skin, these lakes being calcium, barium, aluminum or zirconium salts of acidic dyes such as halo-acid, azo or anthraquinone dyes.

The pearlescent agents may be present in the composition in a proportion of from 0 to 20% by weight, preferably in a content of about 8 to 15% by weight. Among the pearlescent agents which may be envisaged, mention may be made of natural mother-of-pearl, mica coated with titanium oxide, with iron oxide, with natural pigment or with bismuth oxychloride, as well as colored titanium mica.

The fillers, which may be present in a proportion of from 0 to 30% by weight, preferably 5 to 15%, in the composition may be inorganic or synthetic, and lamellar or non-lamellar. Mention may be made of talc, mica, silica, kaolin, Nylon powders, polyethylene powders, Teflon, starch, boron nitride, microspheres such as EXPANCEL (Nobel Industrie), POLYTRAP (Dow Corning) and microbeads of silicone resin (TOSPEARLS from Toshiba for example).

The composition may also comprise any additive commonly used in the cosmetic field, such as antioxidants, fragrances, essential oils, preserving agents, lipophilic and/or hydrophilic cosmetic active agents, moisturizers, vitamins, essential fatty acids, sphingolipids, self-tanning agents such as DHA, and sunscreens.

These additives are found, depending on their solubility, either in the fatty phase or in the phase comprising the polyhydric alcohol or the aqueous phase.

Obviously, a person skilled in the art will take care to select the optional additional compounds, and/or the amount thereof, such that the advantageous properties of the composition according to the invention are not, or are not substantially, adversely affected by the addition envisaged.

The composition according to the invention may also comprise at least one active agent, among which mention may be made of agents that are active against microorganisms, in particular agents with antiviral, antibacterial or antifungal activity; agents with anti-inflammatory or immunomodulatory activity; agents which antagonize neuromediators or modulate the release of neuromediators; agents which modulate cell differentiation and/or proliferation and/or pigmentation and/or which regulate keratinization; agents that are active in the treatment and/or prevention of cheilitis; antihistamines; and cicatrizing agents.

Depending on the pathologies to be treated, it may be prudent to incorporate several active agents, or alternatively to add to them secondary active agents which will enhance the prevention or treatment of the complaint, and/or to treat symptoms associated with this complaint. Among the secondary active agents, mention may be made of local anaesthetics, antiseptics, moisturizers and/or emollients, and sunscreens, in particular chemical or inorganic sunscreens. The composition may also contain an agent for promoting the dissolution or compatibility of the active agents with the excipient of the composition, and/or for promoting the penetration of the active agents into the skin or mucous membranes. This agent may be, for example, isopropyl myristate, oleic acid, lecithin or certain alcohols.

The composition according to the invention may be in the form of a stick or pencil, in the form of an oily liquid, which may optionally be gelled, or alternatively in the form of a soft paste whose viscosity may be measured. The dynamic viscosity at 25° C. is preferably from 3 to 35 Pa·s, measured with a Contraves TV rotary viscometer equipped with an “MS-r4” rotor at a frequency of 60 Hz.

The composition according to the invention may be prepared in the usual manner by those skilled in the art. In particular, the hydrophilic component may be incorporated into the composition by means of the use, during the incorporation, of a turbomixer at a minimum spin speed which produces particles of very small size, in particular smaller than or equal to 1 micron. A large amount of polyhydric alcohol, for example, may thus be incorporated.

In particular, the composition may be prepared by heating all or part of the fatty substances, except for the volatile compounds, and the hydrophilic component to a temperature of from 65 to 95° C., followed by mixing using a turbomixer at a spin speed of at least 1500 revolutions per minute, preferably 2500 to 3500 rev/min.

The volatile compounds and the optional additives liable to be heat-sensitive are then added. The emulsion thus obtained is poured into a suitable mold. The turbomixer which may be used in the process according to the invention may be of any model known commercially for the preparation of cosmetic or pharmaceutical compositions. Mention will be made, for example, of the Moritz turbomixer.

The composition according to the invention may also be prepared with the aid of one or more extruders, which makes it possible in particular to obtain a composition comprising a high content of waxes and being in the form of a soft paste.

The compositions according to the invention may be applied in particular to the field of making up the skin, semi-mucous membranes, mucous membranes and/or the exoskeleton, and are, in this case, for example, in the form of a foundation, a blusher, an eyeshadow, a lipstick, a mascara or an eyeliner.

The compositions may also be in non-colored form, optionally containing cosmetic active agents. In this case, they may be used in particular as a care base for the lips or as a fixing base to be applied to a standard lipstick. In this case, the fixing base forms a protective film over the film of lipstick, limits its transfer and migration and thus increases its staying power.

The compositions according to the invention may also be in the form of a care product for the skin, mucous membranes, semi-mucous membranes and/or the exoskeleton, a hygiene or pharmaceutical product or alternatively an antisun product or self-tanning product.

The invention is illustrated in greater detail in, but not limited by, the following example.

### EXAMPLE 1

A composition was prepared in the form of a lipstick pencil having the following composition:

phenylated silicone oil and PDMS . . . 20%

silicone surfactant . . . 2%

glycerol . . . 10%

waxes (in particular polyethylene waxes) . . . 17%

pigments . . . 8%

cyclotetradimethylsiloxane . . . qs 100%

The silicone oils, the surfactant and the waxes were heated to 95° C.; the pigments were then added. Separately, the glycerol was heated to 80° C.; a Moritz turbomixer at a spin speed of 3000 rev/min was used to mix the fatty phase and the glycerol until a composition of a consistency which was able to be poured was obtained; the volatile compounds were then added and the composition was poured into a suitable mold and left to cool.

A lipstick pencil with a pleasant texture, which spread well and applied uniformly was obtained. The film obtained was very comfortable and moisturizing for the lips. It did not migrate and did not leave traces on external supports after evaporation of the volatile oil (a few minutes). This is all the more noteworthy since the composition comprises a large amount of glycerol.

We claim:

1. A cosmetic, dermatological, hygiene and/or pharmaceutical composition comprising
at least one hydrophilic component and
a fatty phase which comprises at least one volatile compound and at least one phenylated silicone oil.

2. A non-transferring cosmetic, dermatological, hygiene and/or pharmaceutical composition comprising
at least one hydrophilic component and
a fatty phase which comprises at least one volatile compound and at least one phenylated silicone oil.

3. A composition according to claim 1, wherein said at least one hydrophilic component is selected from polyhydric alcohols or is a thickened or gelled aqueous phase.

4. A composition according to claim 1, wherein said at least one hydrophilic component is selected from compounds having from 2 to 8 carbon atoms and from 2 to 6 hydroxyl functions and polyether alcohols with an average molecular weight of 150 to 600.

5. A composition according to claim 4, wherein said compounds having from 2 to 8 carbon atoms and from 2 to 6 hydroxyl functions are selected from ethylene glycol, glycerol, 1,2-propanediol, diglycerol, erythritol, arabitol, adonitol, sorbitol and dulcitol.

6. A composition according to claim 4, wherein said polyether alcohols with an average molecular weight of 150 to 600 are selected from polyethylene glycol 300 and polyglycerol 500.

7. A composition according to claim 3, in which said aqueous phase is thickened using at least one thickener selected from extracts of algae, seed extracts, fruit extracts, plant exudates, cellulose derivatives, gelling agents of animal origin, microorganism exudates, and synthetic gelling agents.

8. A composition according to claim 7, wherein said extracts of algae are selected from agar-agar, carrageenans and alginates, said seed extracts are selected from extracts of carob and extracts of guar, said fruit extract is pectin; said plant exudates are selected from gum arabic, gum tragacanth, karaya gum and ghatti gum, said cellulose derivative is carboxymethylcellulose, said gelling agents of animal origin are selected from gelatin and caseinates, said microorganism exudate is xanthan gum, and said synthetic gelling agents are selected from acrylic polymer derivatives and silicon derivatives.

9. A composition according to claim 7, wherein said at least one thickener is present in the aqueous phase in an amount of from 0.2 to 10% by weight relative to the aqueous phase.

10. A composition according to claim 1, wherein said hydrophilic component represents from 4 to 40% by weight of the composition.

11. A composition according to claim 10, wherein said hydrophilic component represents from 8 to 25% by weight of the composition.

12. A composition according to claim 1, further comprising at least one surfactant.

13. A composition according to claim 1, wherein said at least one volatile compound is selected from hydrocarbon oils, cyclic silicone oils, and linear silicone oils.

14. A composition according to claim 1, wherein said at least one volatile compound is selected from volatile cyclic silicones having from 3 to 8 silicon atoms, dimethylsiloxane/methylalkylsiloxane cyclocopolymers, volatile linear silicones having from 2 to 9 silicon atoms, alkyltrisiloxanes, and volatile hydrocarbon oils.

15. A composition according to claim 14, wherein said volatile cyclic silicones have from 4 to 6 silicon atoms.

16. A composition according to claim 1, wherein said at least one volatile compound represents from 8 to 70% by weight relative to the total weight of the composition.

17. A composition according to claim 16, wherein said at least one volatile compound represents from 15 to 55% by weight relative to the total weight of the composition.

18. A composition according to claim 1, wherein said phenylated silicone oil corresponds to the following formula: 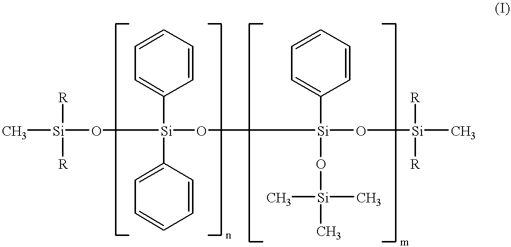
wherein
R is a C₁-C₃₀ alkyl radical, an aryl radical or an aralkyl radical,
n is an integer from 0 to 100,
m is an integer from 0 to 100, with the proviso that the sum m+n is from 1 to 100.

19. A composition according to claim 1, wherein said phenylated silicone oil represents from 1 to 35% by weight relative to the total weight of the composition.

20. A composition according to claim 19, wherein said phenylated silicone oil represents from 20 to 30% by weight relative to the total weight of the composition

21. A composition according to claim 1, further comprising at least one wax.

22. A composition according to claim 21, wherein said at least one wax is selected from microcrystalline waxes, paraffin, petrolatum, petroleum jelly, ozokerite, montan wax, beeswax, lanolin and derivatives thereof, candelilla wax, ouricurry wax, carnauba wax, Japan wax, cocoa butter, cork fibre wax, sugar cane wax; hydrogenated oils which are solid at 25° C., fatty esters and glycerides which are solid at 25° C.; polyethylene waxes, the waxes obtained by Fischer-Tropsch synthesis; silicone waxes; and fluoro waxes.

23. A composition according to claim 21, wherein said wax is present in a proportion of from 0.5 to 30% by weight relative to the total weight of the composition.

24. A composition according to claim 23, wherein said wax is present in a proportion of from 10 to 20% by weight relative to the total weight of the composition.

25. A composition according to claim 1, further comprising a fatty substance selected from oils, gums and pasty fatty substances of plant, mineral, animal, synthetic or silicone origin.

26. A composition according to claim 1, comprising less than 20% by weight of non-volatile hydrocarbon oil.

27. A composition according to claim 26, comprising less than 5% by weight of non-volatile hydrocarbon oil.

28. A composition according to claim 27, comprising no non-volatile hydrocarbon oil.

29. A composition according to claim 1, further comprising a particulate phase comprising pigments and/or fillers and/or pearlescent agents.

30. A composition according to claim 1, further comprising at least one agent selected from agents active against microorganisms, agents with anti-inflammatory or immunomodulatory activity, agents which antagonize neuromediators or which modulate the release of neuromediators, agents which modulate cell differentiation and/or proliferation and/or pigmentation and/or which regulate keratinization, agents active in the treatment and/or prevention of cheilitis, an antihistamine, and a cicatrizing agent.

31. A composition according to claim 1, said composition being in the form of a stick or pencil, in the form of an oily liquid, which is optionally gelled, or in the form of a soft paste with a dynamic viscosity at 25° C. of from 3 to 35 Pa·s, measured with a Contraves TV rotary viscometer equipped with an “MS-r4” rotor at a frequency of 60 Hz.

32. A composition according to claim 1, said composition being in the form of a care and/or make-up product for the skin, mucous membranes, semi-mucous membranes and the exoskeleton; a hygiene product; or a pharmaceutical product.

33. A composition according to claim 32, said composition being in the form of a foundation, a blusher, an eyeshadow, a lipstick, a mascara, an eyeliner, a lipcare product, a fixing base for the lips, a self-tanning product or an antisun product.

34. A process for limiting, decreasing and/or eliminating the transfer of a cosmetic, dermatological, hygiene and/or pharmaceutical composition comprising a hydrophilic component, said process comprising including in said composition at least one volatile compound and at least one phenylated silicone oil.

35. A process according to claim 34, wherein said composition is a make-up or care composition for the skin, mucous membranes, semi-mucous membranes and/or the exoskeleton.
